(19) United States
(12) Patent Application Publication
(10) Pub. No.: US 2024/0390502 A1
(43) Pub. Date: Nov. 28, 2024

(54) DELIVERY SYSTEMS FOR BIOACTIVE LIPIDS

(71) Applicant: The Trustees of Columbia University in The City of New York, New York, NY (US)

(72) Inventors: Richard J. DECKELBAUM, Hastings on Hudson, NY (US); Hylde ZIRPOLI, Long Island City, NY (US); Søren Weis DAHL, New York, NY (US); Jaroslav A. KRALOVEC, Halifax (CA)

(21) Appl. No.: 18/694,195

(22) PCT Filed: Sep. 22, 2022
(86) PCT No.: PCT/US2022/076842

§ 371 Date: Mar. 21, 2024

Related U.S. Application Data
(60) Provisional application No. 63/247,142, filed on Sep. 22, 2021.

(51) Int. Cl. A61K 47/54; A61K 47/59; A61K 47/60; A61P 25/28

(52) U.S. Cl. CPC A61K 47/54; A61K 47/548; A61K 47/59; A61K 47/60; A61P 25/28

(57) ABSTRACT

The invention relates to a bioactive lipid (BAL) composition comprising one or more hydrocarbon-derivatized fatty acids (FAs), which can be an oxygenated, halogenated, amino, amide, nitro, cyano, furanoid, nitroso, isonitrile, or O-substituted derivative of the hydrocarbon-derivatized FA, which are bound to a molecular backbone. In some embodiments, the BALs comprise a specialized pro-resolving mediator (SPMs), eicosanoid, prostanoid, prostaglandin, or endocannabinoid (eCBs) and optionally further comprise omega-3 (n-3) FAs and/or omega-6 (n-6) FAs. In general, the BALs are conjugated to a molecular backbone, preferably a five- or six-carbon monosaccharide, or glycerol. The compositions can be formulated as an emulsion. Preferred BALs can be selected by their performance in one or more cellular assays or animal models. These compositions are useful for tissue or organ protection in a patient, for example in stroke, ischemia, traumatic injury, a neurodegenerative disorder, or organ injury and/or transplant.

### CROSS-REFERENCE TO RELATED APPLICATIONS

This application claims benefit of U.S. Provisional Patent Application No. 63/247,142, filed Jun. 22, 2021, which is incorporated herein in its entirety.

### GOVERNMENT FUNDING SUPPORT

This invention was made with government support under grant nos. NS109717 and NS088197 awarded by the National Institutes of Health. government has certain rights in the invention.

### BACKGROUND OF THE INVENTION

Lipids are the major constituents of cell membranes and act as key mediators of various intercellular and intracellular processes. “Bioactive Lipids” (BAL) play important roles in immune regulation, initiation and resolution of inflammation, and maintenance of cell and tissue homeostasis. BALs include, for example, omega-3 (n-3) and omega-6 (n-6) fatty acids, and their various derivatives such as specialized pro-resolving mediators (SPMs) and eicosanoids. BALs further include endocannabinoids (eCBs) and lysoglycerophospholipids and sphingolipids. Some BALs are generated in vivo from n-6 or n-3 essential polyunsaturated fatty acid (PUFA) precursors that are esterified into membrane lipids. Despite their appreciable potential to impact pathophysiological processes, BALs have not been sufficiently harnessed for their therapeutic benefits.

### SUMMARY OF THE INVENTION

Therefore, there is a need in the art for new bioactive lipid compositions. In particular, the present invention relates, in some embodiments, to a bioactive lipid (BAL) composition comprising one or more BAL which comprises one or more hydrocarbon-derivatized fatty acids (FAs) optionally covalently bound to a molecular backbone. For example, at least one of the BALs is an oxygenated derivative, halogenated derivative, amino derivative, amide derivative, nitro derivative, cyano derivative, furanoid derivative, nitroso derivative, isonitrile derivative, or an O-substituted derivative of the hydrocarbon-derivatized FA and can comprise a specialized pro-resolving mediator (SPMs), eicosanoid, prostanoid, prostaglandin, or endocannabinoid (eCBs) and optionally further comprise omega-3 (n-3) FAs and/or omega-6 (n-6) FAs. In certain embodiments, the SPM is conjugated or esterified to the molecular backbone of the composition and the omega-3 FA is conjugated or esterified to the molecular backbone, and can be derived from EPA, DHA, DPA, or arachidonic acid.

In particular embodiments, the at least one SPM is selected from Resolvin D1, Resolvin D2, Resolvin D3, Resolvin D4, Resolvin D5, Resolvin D6, Resolvin E1, Resolvin E2, Resolvin E3, Protectin D1, Resolvin T1, Resolvin T2, Resolvin T3, Resolvin T4, Resolvin 1 n-3 DPA, Resolvin 2 n-3 DPA, Resolvin 5 n-3 DPA, Protectin 1 n-3 DPA, Protectin 2 n-3 DPA, Maresin 1, Maresin 2, Maresin 1 n-3 DPA, Maresin 2 n-3 DPA, and Maresin 3 n-3 DPA, Lipoxin A₄, Lipoxin B₄, or a stereoisomer thereof.

The BAL composition can comprise at least two SPMs independently selected from SPMs derived from EPA, SPMs derived from DHA, and SPMs derived from DPA or can comprise at least two, at least three, or at least four SPMs independently selected from Resolvin E1, Resolvin E2, Resolvin E3, Resolvin D1, Resolvin D2, Resolvin D3, Resolvin D4, Resolvin D5, Resolvin D6, Protectin D1, Maresin 1, Maresin 2, Resolvin T1, Resolvin T2, Resolvin T3, Resolvin T4, Resolvin 1 n-3 DPA, Resolvin 2 n-3 DPA, Resolvin 5 n-3 DPA, Protectin 1 n-3 DPA, Protectin 2 n-3 DPA, Maresin 1 n-3 DPA, Maresin 2 n-3 DPA, and Maresin 3 n-3 DPA.

In some embodiments, the BAL composition can comprise prostaglandins selected from PDG₂, PGE₂, PGI₂, and PGF₂α and the prostanoid is selected from prostacyclin and hromboxane. The compounds also can further comprise one or more hydroxyeicosatetraenoids (HETEs), which can be selected from leukotrienes and lipoxins (LX). In certain embodiments, the BAL composition can further comprise epoxyeicosatrienoids, such as arachidonoylethanolamide (AEA), 2-arachidonoylglycerol (2-AG), 2-AG-ether, O-arachidonoylethanolamine, and palmitoylethanolamide (PEA).

In some embodiments, the BAL composition comprises at least about 50% n-3 FAs or at least about 75% n-3 FAs.

In some embodiments, the BAL composition comprises one or more of docosahexaenoic acid (DHA) and/or eicosapentaenoic acid (EPA) and/or docosapentaenoic acid (DPA) esterified to the molecular backbone.

In preferred embodiments, the molecular backbone of the BAL composition is glycerol. In certain embodiments, the BAL composition is substantially diglyceride or substantially triglyceride. The molecular backbone also can comprise phosphoglycerol, phosphatidylcholine, phosphatidylserine, phosphatidylinositol, and/or phosphatidylethanolamine. In certain embodiments, the molecular backbone of the BAL composition comprises a five-carbon or six-carbon monosaccharide. In certain embodiments, the molecular backbone of the BAL composition is a polymer, optionally a hydroxyl-containing polymer, and optionally polyethylene glycol, poly(dimethylamine-co-epichlorohydrin, poly(ethyleneglycol) diamine, and/or polyethylenimine or is an amino alcohol.

In certain embodiments, the BAL composition is formulated as an emulsion, for example an oil-in-water emulsion having a mean particle size of 200 nm or less. In some embodiments, the BAL composition comprises at least about 5% by weight of the emulsion.

These emulsions preferably comprise one or more emulsifiers such as one or more of a phospholipid emulsifier, a phosphatidylcholine emulsifier and one or more medium chain or long chain FAs emulsifiers.

Embodiments of the invention also include a method for making the BAL compositions comprising, performing a condensation reaction with the molecular backbone and one or more BAL, which may be enzyme-catalyzed, optionally with a lipase. In some embodiments, the condensation reaction is an esterification or transesterification reaction.

In a preferred embodiments, the method comprises: selecting a combination of BALs for therapeutic delivery; conjugating the BALs to a molecular backbone to form a BAL composition; and formulating the BAL composition as an emulsion suitable for delivery to a subject in need.

In preferred embodiments, the BALs are selected by their performance in one or more cellular assays selected from one or more of evaluating caspase-3/7 activation, MTT cell viability assay, lactate dehydrogenase (LDH) assay, TUNEL assay, Annexin V/propidium iodide (PI) assay, detection of proteins associated with apoptosis or cell death, cytokine detection, assays for oxidative stress or ROS production, assays for inflammasome activation, expression or production of mitochondrial biomarkers, measurement of intracellular Ca2+, and/or one or more animal models oxygen and glucose deprivation (OGD) in vitro, selected from middle cerebral artery occlusion (MCAo), hypoxic-ischemic injury, LPS-induced cell inflammation in vitro, traumatic brain injury, left anterior descending artery (LAD) occlusion, global heart ischemia ex vivo system, and spinal cord injury in vivo.

Embodiments of the invention also include a method for tissue or organ protection in a patient, comprising administering an effective amount of the compositions described herein to a patient in need. Suitable patients include those wherein the patient is at risk of ischemia reperfusion injury, the patient is experiencing stroke, the patient is recovering from stroke, the patient is at risk of stroke, the patient has hypoxic-ischemic encephalopathy, the patient is at risk for hypoxic-ischemic encephalopathy, the patient is experiencing myocardial infarction, the patient is recovering from myocardial infarction, the patient is at risk of myocardial infarction, the patient is suffering from traumatic brain injury, the patient is at risk of traumatic brain injury, the patient is suffering from post-traumatic stress disorder, the patient is suffering from a spinal cord injury, the patient is suffering from a neurodegenerative disease, the patient is suffering from acute organ injury, or the patient is an organ transplant recipient. The stroke can be ischemic stroke, hemorrhagic stroke, or neonatal stroke. The neurodegenerative disease is amyotrophic lateral sclerosis, multiple sclerosis, Parkinson's disease, Alzheimer's disease, Dementia, or Huntington's disease.

Embodiments of the invention also include method wherein the composition is administered intravenously, intra-arterially, intrathecally, or by intragastric or intraduodenal tube. In certain embodiments, the patient is administered the composition from 1 to 10 times, with frequencies ranging from about once every four hours to about once per week.

### BRIEF SUMMARY OF THE DRAWINGS

FIG. 1A and FIG. 1B are a table showing specialized pro-resolving mediators.

FIGS. 2A-C shows that NPD1 treatment induced significant reduction in cerebral infarct volumes upon direct administration after hypoxic-ischemic injury in a mouse model, and this was associated with both preserved mitochondrial Ca2+ buffering capacity and reduced mitochondria-related cell death pathways. FIG. 2A presents infarct volumes in saline (n=15), 10 ng NPD1 (n=16), and 20 ng NPD1 (n=14). FIG. 2B is a photograph showing representative of TTC-stained cerebral sections from each group. FIG. 2C is a western blot mean quantification for BAX 6A7 in naïve (n=6), saline (n=7) and NPD1 (n=8) at 4 h after ischemic injury.

FIG. 3 shows infarct volumes in neonatal mice subjected to hypoxic-ischemic injury and treated with saline as vehicle, n-3 triglyceride (TG), or n-3 diglyceride (DG) emulsions. N=15-17. Values are means±SD. Dose is 2×0.375 mg/g body weight, one hour apart.

FIG. 4 shows average infarct volumes in mice treated immediately after ischemic with either saline, DHA, EPA, DHA+EPA, or ARA (all DG emulsions).

FIG. 5 is a schematic illustrating a platform for discovery of novel DG therapeutics.

FIG. 6 illustrates the composition of DGs according to embodiments of the invention.

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about” means plus or minus 10 percent of the recited value.

### 2. Embodiments of the Invention

In the various aspects and embodiments, the present disclosure relates to compositions comprising at least one bioactive lipid (BAL) attached to a molecular backbone, as well as methods for discovering and making such compositions, and methods for therapy involving the same. The compositions described herein are useful for acute and chronic therapy to treat and/or prevent tissue or organ injuries. In various embodiments, the compositions provide protection from cellular death, and find use in patients in need of organ protection, including neuroprotection or cardio protection. For example, the compositions find use in treating ischemia reperfusion injuries, such as ischemic stroke and myocardial infarction. The compositions further find use for treatment of traumatic injuries, such as traumatic brain injury or spinal cord injury, among others. In other embodiments, the compositions find use for treating, preventing, or slowing the progression of neurodegenerative diseases or injuries.

The invention in various aspects and embodiments relates to compositions comprising BALs attached to a molecular backbone, including but not limited to a glycerol backbone. BALs that find use with the invention can include natural fatty acids (FAs) and natural and synthetic derivatives (including but not limited to derivatives of n-3 and n-6 FAs), including hydrocarbon-derivatized FAs such as oxygenated derivatives (including hydroxylated, carbonyl, and epoxy derivatives), halogenated derivatives, amino derivatives, nitro derivatives, cyano derivatives, furanoid derivatives, nitroso derivatives (C- or S-), isonitrile derivatives, and O-substituted derivatives (e.g., alkoxy substituted), among others.

BALs can further include amide derivatives, including primary, secondary, and tertiary amides. Exemplary BALs include n-3 FAs, n-6 FAs, specialized pro-resolving mediators (SPMs), eicosanoids, and endocannabinoids (eCBs). Such BALs (either individually or in various combinations) can be conjugated to a molecular backbone such as glycerol (i.e., forming monoglycerides, diglycerides, and/or triglycerides), or other molecular backbones. In some embodiments, the compositions can include other molecules conjugated to the molecular backbone in addition to BALs.

In some embodiments, the compositions comprise one or more n-3 FAs or n-6 FAs, optionally with one or more other BALs, which in some embodiments are natural or synthetic derivatives of n-3 or n-6 FAs. n-3 FAs are polyunsaturated FAs where one of the carbon-carbon double bonds is between the third and fourth carbon atoms from the distal end of the hydrocarbon chain. Examples of n-3 FAs include α-linolenic acid (18:3n-3; α-ALA; A3,6,9), eicosapentaenoic acid (20:5n-3; EPA; Δ5,8,11,14,17), docosahexaenoic acid (22:6n-3; DHA; Δ4, 7,10,13,16,19) and docosapentaenoic acid (22:5n-3; DPA; Δ7,10,13,16,19). n-3 FAs having at least 20 carbon atoms are referred to as “long chain n-3 FAs”. n-6 FAs include arachidonic acid (AA). Sources of n-3 FAs include fish oils, algae oils, microbial and other oils. n-3 and n-6 FAs may also be synthesized. In some embodiments, the BALs do not comprise EPA, DHA, DPA, or AA, or comprise at least one BAL other than EPA, DHA, DPA, and AA.

In some embodiments, the BALs comprise one or more eiscosanoids. The eicosanoids include molecules that have AA as their biosynthetic precursor, and are often considered initiators of inflammation, among other biological properties. For example, AA acts as a substrate for several oxygen-incorporating enzymes synthesizing numerous eicosanoids, including: prostanoids which include prostaglandins (PGs, such as PDG₂, PGE₂, PGI₂, and PGF_{2α}), prostacyclins, and thromboxanes (TXA₂ and TXB₂); hydroxyeicosatetraenoids (HETEs) which include leukotrienes (LTs) and lipoxins (LX); and epoxyeicosatrienoids.

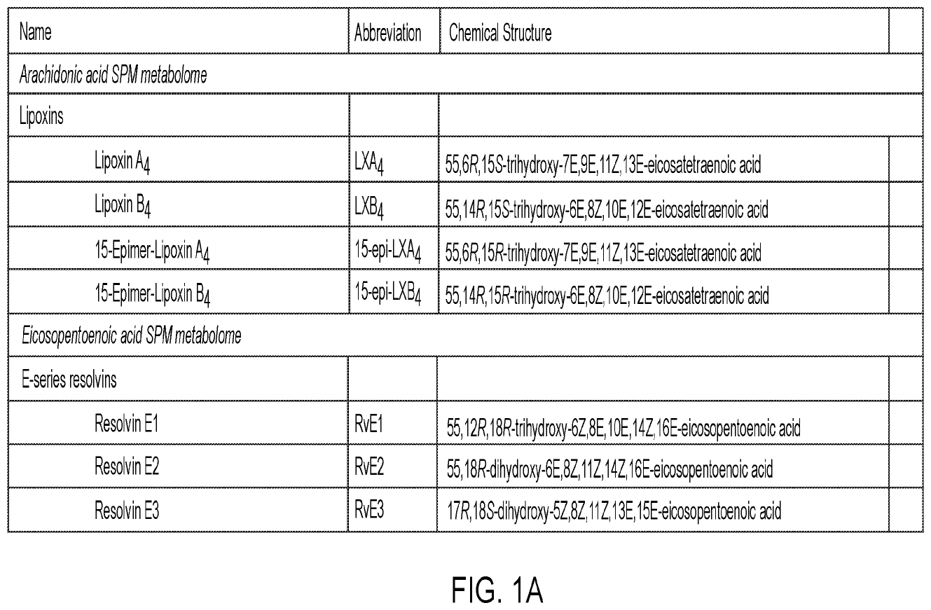

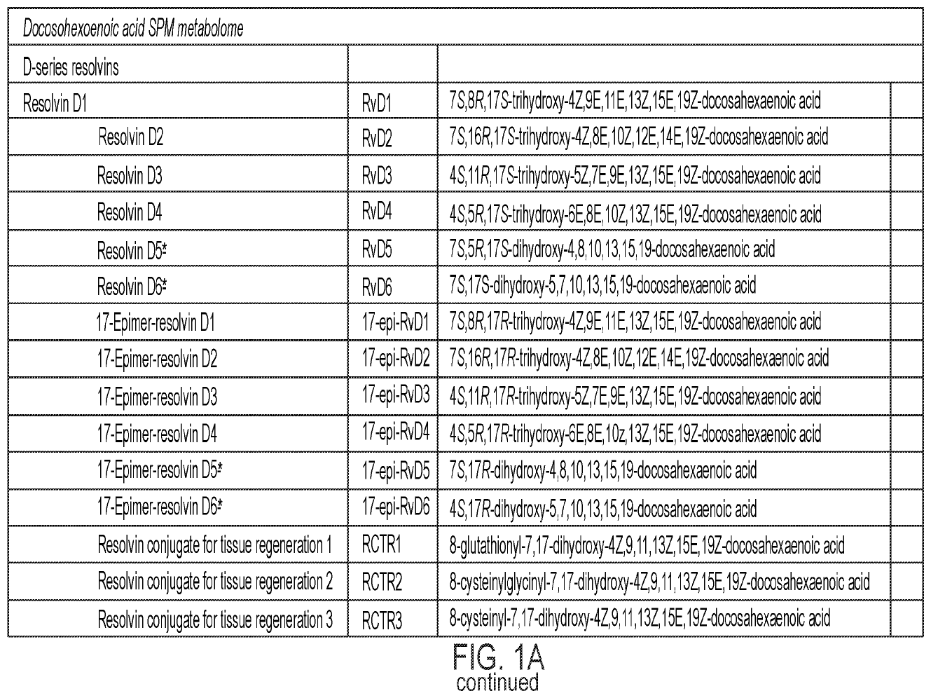

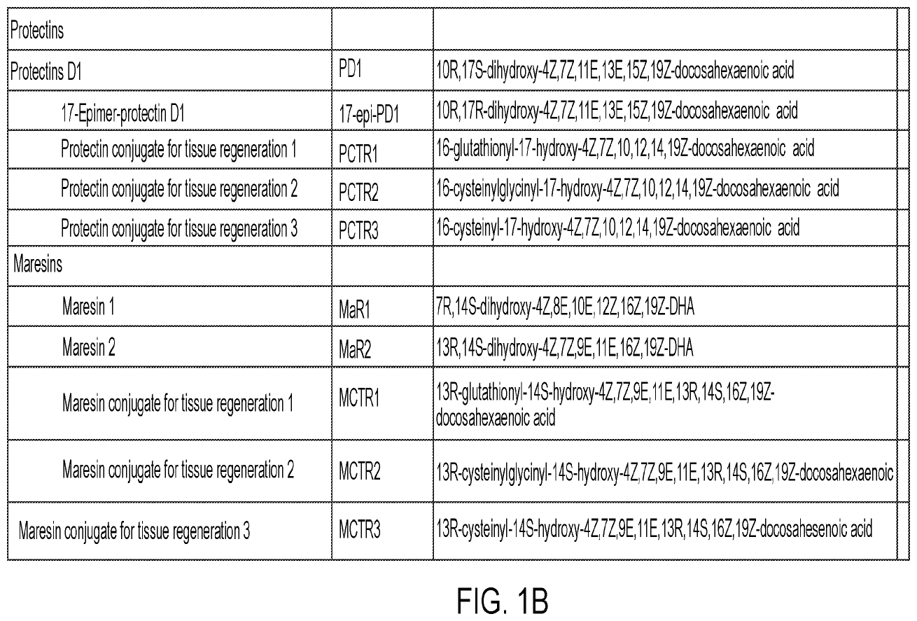

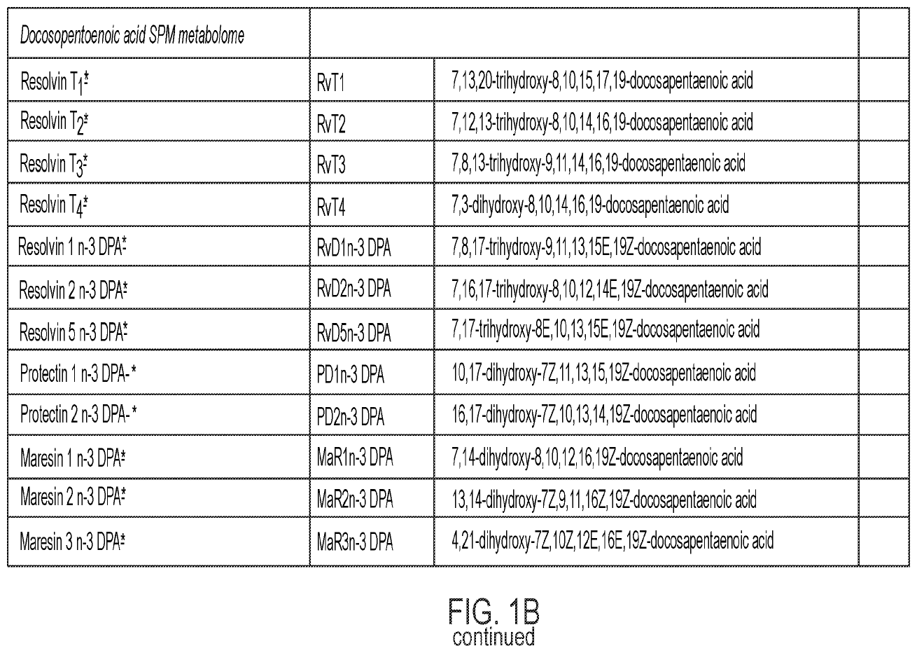

In some embodiments, the BALs comprise one or more SPMs. The SPMs are oxygenated metabolites derived mainly from AA, EPA, DPA, and DHA. They include lipoxins, (neuro)protectins, resolvins, and maresins and can have potent anti-apoptotic, anti-inflammatory and immunoregulatory effects at concentrations in the nanomolar to picomolar range. SPMs are produced by dioxygen-dependent oxidation from their n-3 FA and n-6 FA precursors. SPMs include certain AA-derived lipoxins (LXA₄ and LXB₄), EPA-derived E-series resolvins (RvE₁₋₃), DHA-derived D-series resolvins (RvD₁₋₆), protectins/neuroprotectins (PD1/NPD1 and PDX), maresins (MaR₁ and MaR₂), and DPA-derived 13-series resolvins (RvT₁₋₄). SPMs can act as immunoresolvents. SPMs are summarized in the table in FIG. 1.

In some embodiments, the BALs comprise one or more endocannabinoids (eCBs). eCBs include a group of BALs that activate the same receptors as the main psychoactive component of marijuana, and include Arachidonoylethanolamide (AEA), 2-arachidonoylglycerol (2-AG), 2-AG-ether, O-arachidonoylethanolamine, and palmitoylethanolamide (PEA), as well as natural and synthetic derivatives of these (including oxygenated derivatives). eCBs are potent immunoregulatory compounds, capable of regulating the functions of several cell subsets of innate or adaptive immunity.

The BALs are conjugated to a molecular backbone such as glycerol. Thus, in some embodiments, the composition comprises one or a mixture of monoglycerides, diglycerides, and triglycerides. In still other embodiments, the molecular backbone is phosphoglycerol (thus forming glycerophospholipids or lysoglycerophospholipids with BALs), or sphingosine (forming sphingolipids such as a ceramide or sphingomyelin with one or more BALs). In some embodiments, the molecular backbone may further comprise polar head groups, such as ethanolamine, phosphoethanolamine, choline, phosphocholine, inositol, phosphoinositol, and amino acids such as serine or phosphoserine. In some embodiments, the BALs are conjugated or esterified to glycerol to prepare diglyceride oils or emulsions thereof.

A number of biological mechanisms can be affected by BALs and which can be beneficial in acute injury, including (i) decrease in generation of mitochondrial ROS; (ii) preservation of mitochondrial Ca2+ uptake and homeostasis; (iii) modulation of receptor-mediated signal transduction and regulation of apoptotic pathways; (iv) decrease in inflammatory responses. Working separately or synergistically, these mechanisms can contribute to, for example, protection in ischemic injury, inhibition of cell death pathways, while accelerating repair processes.

In some embodiments, at least one BAL is derived from EPA and belonging to the family of SPMs. SPMs derived from EPA are shown in FIG. 1. For example, at least one SPM may be selected from Resolvin E1, Resolvin E2, and Resolvin E3.

In some embodiments, at least one BAL is derived from DHA and belonging to the family of SMPs. SPMs derived from DHA are shown in FIG. 1. For example, at least one SPM may be selected from Resolvin D1, Resolvin D2, Resolvin D3, Resolvin D4, Resolvin D5, Resolvin D6, or a stereoisomer thereof. In some embodiments, at least one SPM is Protectin D1 or a stereoisomer thereof. In some embodiments, at least one SPM is Maresin 1 or Maresin 2.

In some embodiments, at least one BAL is derived from DPA and belonging to the family of SMPs. SPMs derived from DPA are shown in FIG. 1. For example, at least one SPM may be selected from Resolvin T1, Resolvin T2, Resolvin T3, Resolvin T4, Resolvin 1 n-3 DPA, Resolvin 2 n-3 DPA, Resolvin 5 n-3 DPA, Protectin 1 n-3 DPA, Protectin 2 n-3 DPA, Maresin 1 n-3 DPA, Maresin 2 n-3 DPA, and Maresin 3 n-3 DPA.

In some embodiments, at least one BAL is derived from arachidonic acid (AA) and belonging to the family of SMPs. SPMs derived from AA are shown in FIG. 1. For example, at least one such SPM may be selected from Lipoxin A₄, Lipoxin B₄, or a stereoisomer thereof.

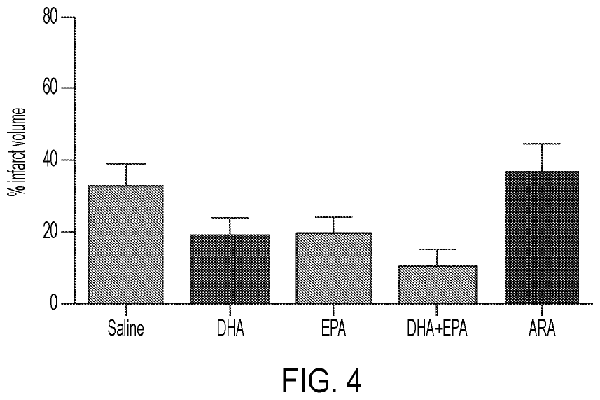

In some embodiments, the composition comprises at least two SPMs independently selected from SPMs derived from EPA, SPMs derived from DHA, and SPMs derived from DPA. For example, the composition may comprise at least two, at least three, or at least four SPMs independently selected from Resolvin E1, Resolvin E2, Resolvin E3, Resolvin D1, Resolvin D2, Resolvin D3, Resolvin D4, Resolvin D5, Resolvin D6, Protectin D1, Maresin 1, Maresin 2, Resolvin T1, Resolvin T2, Resolvin T3, Resolvin T4, Resolvin 1 n-3 DPA, Resolvin 2 n-3 DPA, Resolvin 5 n-3 DPA, Protectin 1 n-3 DPA, Protectin 2 n-3 DPA, Maresin 1 n-3 DPA, Maresin 2 n-3 DPA, and Maresin 3 n-3 DPA. In some embodiments, the composition comprises one or more SPMs derived from EPA (as described above), and includes one or more SPMs derived from DHA (as described above). It is believed that these combinations can provide for synergistic effects, as demonstrated in FIG. 4 for the delivery of EPA and DHA via diglyceride emulsions in an ischemic injury model. For FIG. 4, all emulsions are dosed at 0.375 g glyceride/kg.

In some aspects and embodiments, the present disclosure provides a method for making compositions that deliver one or combinations of BALs for treating or preventing an organ or tissue injury or for modulating inflammatory or apoptotic processes. In various embodiments, the method comprises testing BALs (either individually or in combination) in cellular assays. The assays are selected based on the intended use of the composition, for example, for inhibiting processes involved in ischemic organ injury, myocardial or cardiovascular injury, traumatic organ or tissue injury, or neurodegenerative disease, for example. In some embodiments, combinations of BALs are tested to identify synergistic combinations. In some embodiments, optimal ratios or concentrations of selected BALs are determined in the cellular assays or in vivo models. Once one or a combination of BALs are selected, the BAL(s) are conjugated to molecular backbones to facilitate delivery, including glycerol backbones to form monoglyceride (MG) oils, diglyceride (DG) oils, and/or triglyceride (TG) oils and emulsions thereof. Biological activity of the oils or emulsions can be validated with in vitro (cell-based assays) as well as with in vivo animal models.

Thus, the selection and relative amounts of BALs to be delivered may be informed by in vitro cellular assays. In some embodiments, the assays can measure apoptotic processes.

For example, apoptosis initiated via intrinsic or extrinsic pathways, and inhibition of apoptosis by BALs, can be detected by measuring caspase-3/7 activation in a cell line, such as a neuronal cell line. Other exemplary assays for evaluating inhibition of apoptosis and cytoprotective properties of BALs include MTT cell viability assay, Lactate dehydrogenase (LDH) assay, TUNEL assay, Annexin V/propidium iodide (PI) assay, and detection of proteins associated with apoptosis or other cell death pathways (e.g., using Western blot), among others. Additional useful assays to measure the physiological state of the cell include assays for cytokine detection (e.g., ELISA), assays for oxidative stress or ROS production, assays for inflammasome activation, assay for mitochondrial functionality and dynamics, and measurement of intracellular Ca²⁺. Cell lines can be selected based on the organ or tissue intended for treatment, and can include epithelial and endothelial cell lines, as well as hematopoietic cells. Exemplary cell lines include but are not limited to cardiac muscle cell lines, brain cell lines (such as neuronal or glial cell lines), hepatocyte cell lines, renal cell lines, colon epithelial cell lines, endothelial cell lines, and immune cell lines (e.g., macrophage or dendritic cell lines), among others.

Various in vivo and in vitro models for evaluating effects of BAL(s) or compositions thereof include: ischemia-reperfusion in vitro, using oxygen and glucose deprivation (OGD), middle cerebral artery occlusion (MCAo) as a model of ischemic stroke, hypoxic-ischemic injury (as a model of HI encephalopathy), LPS-induced cell inflammation (in vitro and in vivo model), traumatic brain injury (single or repetitive models), left anterior descending artery (LAD) occlusion (e.g., as a model for myocardial infarction), global heart ischemia (ex vivo system), spinal cord injury (in vivo), as well as neurodegenerative models which are known in the art.

In various embodiments, a BAL oil (e.g., MG, DG, or TG) comprises up to about 50% of BALs by weight of the composition. In some embodiments, a BAL oil comprises at least about 0.01%, or at least about 0.1%, or at least about 1%, or at least about 5%, or at least about 10%, or at least about 20% of BALs by weight of the composition. In some embodiments, the composition comprises from about 0.1% to about 25% BALs by weight, or from about 1% to about 20% BALs by weight, or from about 5% to about 15% BALs by weight of the composition.

In various embodiments, the composition comprises one or more omega-3 fatty acids conjugated or esterified to the molecular backbone, with one or more additional BALs such as one or more SPMs. In such embodiments, the composition may comprise at least about 50% omega (n-3) fatty acids (FAs) or at least about 75% n-3 FAs, or at least about 90% n-3 FAs, by weight of the total BALs esterified or conjugated to the molecular backbone. For example, in various embodiments, the composition comprises from about 70% to about 90% n-3 FAs by weight of BALs conjugated or esterified to the molecular backbone. For example, the composition may comprise one or more of DHA, EPA, and/or DPA, esterified or conjugated to the molecular backbone. In some embodiments, the composition comprises EPA and DHA esterified or conjugated to the molecular backbone.

In accordance with embodiments of the invention, the FAs of the composition may be predominately n-3 FAs and SPMs. In various embodiments, the composition comprises at least about 50% n-3 FAs and SPMs, or at least about 75% n-3 FAs and SPMs, or at least about 90% n-3 FAs and SPMs, or at least 95% n-3 FAs or SPMs, or about 100% n-3 FAs and SPMs. In some embodiments, the n-3 FAs are long chain n-3 FAs, including one or more of DHA, EPA, and DPA. In some embodiments, the n-3 FAs comprise DHA. In some embodiments, the n-3 FAs are at least about 50% DHA, or at least about 60% DHA, or at least about 75% DHA. In some embodiments, the n-3 FAs comprise EPA. For example, the n-3 FAs may be at least about 50% EPA, or at least about 60% EPA, or at least about 75% EPA. In some embodiments, the n-3 FAs comprise DPA. For example, the n-3 FAs may be at least about 50% DPA, or at least about 60% DPA, or at least about 75% DPA. In some embodiments, the n-3 FAs comprise DHA and EPA, which are optionally present at a ratio of from about 2:1 to about 1:2 (e.g., about 1:1). As demonstrated herein, DG emulsions carrying DHA+EPA show exceptionally high properties in neuroprotection. See FIG. 4.

The composition may comprise other lipophilic active agents, either conjugated to the molecular backbone or incorporated in emulsions, to enhance their delivery and provide synergistic results with other mechanisms of action. For example, in some embodiments the compositions comprise glibenclamide, lipophilic statins (e.g., atorvastatin, rosuvastatin, fluvastatin, lovastatin, simvastatin or cerivastatin), lipophilic neuroprotectants (e.g., 170-Estradiol, simvastatin, or progesterone), corticosteroids, cannabinoids (e.g., tetrahydrocannabinolic acid (THCA), tetrahydrocannabinol (THC), cannabidolic acid (CBDA), cannabidiol (CBD), cannabinol (CBN), cannabigerol (CBG), cannabichromene (CBC), and tetrahydrocannabivarin (THCV)), stilbenoids (e.g., resveratrol), and others, can be incorporated into the compositions.

In various embodiments, the molecular backbone is glycerol, with the BALs and FAs (and optionally other lipophilic compounds) esterified thereto. In some embodiments, the composition comprises one or a mixture of monoglycerides, diglycerides, and triglycerides. In some embodiments, the composition in various embodiments may be substantially or predominately (e.g., more than about 50% or more than about 75%, or more than about 90% by weight) diglyceride (DG), with respect to the total amount of glycerides. Alternatively, the composition may be substantially or predominately (e.g., more than about 50%, or more than about 75%, or more than about 90% by weight) triglyceride (TG), with respect to the total amount of glycerides.

According to this disclosure, DGs are defined as comprising two FAs, BALs, or other lipophilic molecule esterified to the trihydric alcohol glycerol. An exemplary method for synthesis of DG molecules is through lipase-catalyzed glycerolysis (i.e., transesterification) with BALs. In various embodiments, the compositions described herein are substantially DG, that is, such compositions do not contain large amounts of triglycerides. In some embodiments, the emulsion compositions are at least about 75%, or at least about 85%, or at least about 90%, or at least about 95% DGs, with respect to the total amount of glycerides present in the composition. In various embodiments, the DG molecules comprise 1,3-DGs and 1,2-DGs. In some embodiments, the DGs are predominately (greater than about 50% or greater than about 75%) 1,3-DGs.

In other embodiments, various derivatives of glycerol may be employed as the molecular backbone, such as but not limited to phosphoglycerol, phosphatidylcholine, phosphatidylserine, phosphatidylinositol, and phosphatidylethanolamine. In these embodiments, one or two moieties as described herein (BALs or other lipophilic compounds) can be esterified to the backbone.

In still other embodiments, the molecular backbone is a five-carbon or six-carbon monosaccharide, which can increase the number of moieties delivered simultaneously. In still other embodiments, the molecular backbone is a natural or synthetic polymer. Exemplary polymers include hydroxyl-containing polymers, such as but not limited to polyethylene glycol, hydroxylethyl acrylate, among many others including poly(dimethylamine)-co-epichlorohydrin, poly(ethyleneglycol) diamine, polyethylenimine. Other hydroxyl-containing backbones may be used, including but not limited to amino alcohols.

In various aspects and embodiments, the present invention provides compositions of oil-in-water emulsions comprising the BAL compositions, including but not limited to DG emulsions. In various aspects and embodiments, the compositions are stable emulsions that can be stored in stable form for use in the emergency setting. For example, in various embodiments, the compositions will be delivered on-site by emergency medicine professionals. The emulsions may be substantially stable for at least six months, or at least one year, or at least 18 months, or at least two years, in various embodiments. The compositions are suitable for parenteral delivery routes, such as intravenous or intra-arterial delivery, or other routes including but not limited to intrathecal and enteral delivery. Further, in some embodiments, the physical properties of the emulsions, such as mean particle diameters of 200 nm or less, facilitate delivery of the biolipids to, and/or uptake by, brain tissue.

In various embodiments, the emulsions (including DG emulsions) have a mean particle size of 200 nm or less. In some embodiments, the mean particle size of the emulsions is about 190 nm or less, or about 180 nm or less, or about 170 nm or less, or about 160 nm or less, or about 150 nm or less, or about 140 nm or less, or about 120 nm or less, or about 100 nm or less, or about 90 nm or less, or about 80 nm or less. In some embodiments, the mean particle size is about 140 nm, about 120 nm, or about 110 nm, or about 100 nm, and with a polydispersity index of less than about 0.3, or less than about 0.25, or less than about 0.2. In some embodiments, the mean particle size is from about 110 nm to about 180 nm, or from about 120 nm to about 180 nm, with a polydispersity index of less than about 0.3. In various embodiments, the zeta potential of the emulsions is at least as negative as about −35 mV, or at least as negative as about −40 mV, or at least as negative as about −50 mV, or at least as negative as about −55 mV. The emulsions in accordance with these embodiments are stable, meaning these parameters are maintained for at least six months, or in some embodiments, at least one year, at least 18 months, or at least two years. In accordance with this disclosure, stability is determined with storage at 4° C.

In some embodiments, the stable emulsions are suitable for parenteral administration (e.g., i.v.), to rapidly deliver the bioactive lipids to injured tissues, including the brain or heart. Thus, in such embodiments the composition is an injectable composition. The lipid component of the emulsion will generally be from about 5% to about 50% by weight of the emulsion. In some embodiments, the lipid component of the composition will be about 10% to about 30%, or about 15% to about 25%. In some embodiments, the lipid component is from 20% to about 40% by weight, or from about 20% to about 30% by weight. For example, the lipid component may be at least about 10%, or at least about 15%, or at least about 20% of the composition by weight, or at least about 25% of the composition by weight, or at least about 30% of the composition by weight. In such embodiments, at least about 10% by weight of the composition is DG oil, or at least about 15% by weight of the composition is DG oil, or at least about 20% by weight of the composition is DG oil, or at least about 23% by weight of the composition is DG oil, or at least about 25% by weight of the composition is DG oil, or at least about 27% by weight of the composition is DG, or at least about 30% by weight of the composition is DG oil. In some embodiments, the composition is about 10 wt. % DG oil. In some embodiments, the composition is from 22 to 27 wt. % DG oil.

Polydispersity index (PDI) is a measure of particle size distribution within a given sample. The numerical value of PDI ranges from 0.0 (for a sample with perfectly uniform particle size distribution) to 1.0 (for a highly polydisperse sample with multiple particle size populations). In lipid-based carriers, such as emulsions, a PDI of 0.30 is desired, indicating a sufficiently homogenous particle size distribution. In some embodiments, the PDI of the emulsions is less than about 0.30, such as about 0.25 or less, 0.20 or less, or about 0.15 or less.

The compositions will comprise one or more emulsifiers to obtain the desired physical characteristics of the emulsion. In various embodiments, emulsifiers can include one or more of phospholipid emulsifiers, phosphoglyceride emulsifiers, and medium and/or long chain fatty acid emulsifiers. In various embodiments, the composition comprises from about 0.5% to about 2.4% by weight of emulsifiers (e.g., phospholipid emulsifiers), such as from about 0.5% to about 2%, and optionally less than about 1.0% by weight of emulsifiers, and optionally from 0.5% to 0.8% of emulsifiers by weight (e.g., phospholipid emulsifiers).

In some embodiments, emulsions comprise one or more phospholipid emulsifiers and/or one or more phosphoglyceride emulsifiers. Phosphoglyceride emulsifiers may be selected from phosphatidylcholine, phosphatidylethanolamine, phosphatidylinositol, phosphatidylserine, and phosphatidic acid. In some embodiments, the composition comprises a phosphatidylcholine emulsifier. In various embodiments, the ratio of phospholipid and/or phosphoglyceride emulsifier to DG (by weight) is 1:8 or less, or is 1:10 or less, or is 1:12 or less, or is 1:15 or less. In some embodiments, the emulsifier comprises at least about 70% phosphatidylcholine, or comprises at least about 80% phosphatidylcholine. For example, the emulsifier (with any co-emulsifier) may contain from about 60% to about 80% phosphatidylcholine.

The composition may further comprise one or more of medium chain or long chain FAs as co-emulsifier, or as components of the glyceride. For example, the composition may comprise a long chain FA, optionally selected from a C16 to C24 FA, and which is optionally a C18 FA. In some embodiments, the co-emulsifier comprises a saturated FA, optionally selected from lauric acid, myristic acid, palmitic acid, and stearic acid. In some embodiments, the co-emulsifier comprises an unsaturated FA, optionally selected from oleic acid or linolenic acid. The co-emulsifier may be added as an alkali metal salt, which optionally comprises sodium oleate. In exemplary embodiments, the co-emulsifier is present at about 0.01% to 5% of the total weight of the composition. For example, the co-emulsifier may be present at from about 0.01 to 2% of the total weight of the composition, or from about 0.01% to about 1% of the total weight of the composition, or from about 0.01% to about 0.05% by weight of the composition.

In various embodiments, the composition is approximately isotonic with human blood, and optionally comprises one or more polyols, such as glycerol, sorbitol, xylitol, and/or glucose. For example, the composition may comprise glycerol from about 2% to about 10% by weight of the composition, or from about 2% to about 7% by weight of the composition.

In some embodiments, the composition comprises one or more anti-oxidants, such as one or more of α-tocopherol, β-tocopherol, γ-tocopherol, δ-tocopherol and an ascorbyl ester. In exemplary embodiments, the anti-oxidants comprise α-tocopherol and/or ascorbyl ester, which is optionally ascorbyl palmitate.

In some embodiments, the composition comprises a metal chelating agent, which is optionally EDTA or EGTA. For example, emulsions may contain from about 5 mM to about 15 mM EDTA or EGTA. For example, in some embodiments, the emulsions contain about 10 mM EDTA.

In various embodiments, emulsions can be prepared according to a process comprising: (1) preparing a mixture of water, glycerol, and EDTA having a temperature of from about 50° C. to about 80° C. (e.g., about 60° C.); (2) add phosphatidylcholine emulsifier (e.g., at least about 75% PC, which may be from egg yolk lecithin), co-emulsifier (e.g., sodium oleate), and bioactive lipid composition (e.g., in the form of a DG oil); (3) homogenize at a temperature of from about 50° C. to about 80° C. (e.g., about 60° C.); (4) process through a microfluidizer or for larger volumes, a high pressure homogenizer (i.e., a high shear fluid processor). The pressure applied during this process could range from 300 to 2000 bar, and in some embodiments, from about 500 to about 1000 bar, such as from about 600 to about 1000 bar. For example, the mixture can be processed through the microfluidizer at about 950-bar pressure at about 60° C. The emulsions can be processed for a length of time and under conditions required to meet the target particle size. This process can include co-formulation of other lipophilic agents as described.

In various embodiments, the pH of the composition is from about 6 to about 10, and optionally from about 6.5 to about 10, and optionally from about 9 to about 10 (e.g., 9.5).

In various embodiments, the composition (for single injection) has a volume of about 500 mL or less, or a volume of about 300 mL or less, or a volume of about 100 mL or less, or a volume of about 50 mL or less, or a volume of about 25 mL or less. In various embodiments, the composition is contained in a pre-filled syringe, optionally having a volume for injection of from about 1 mL to about 50 mL. In some embodiments, the composition is packaged in vials at a volume of from about 25 mL to about 100 mL.

In other aspects, the invention provides a method for making a composition of the present disclosure. In various embodiments, the method comprises providing the at least one BAL optionally with other fatty acids or esters thereof (or other lipophilic compounds) as described, and performing a condensation reaction with the desired molecular backbone (including but not limited to glycerol). In some embodiments, the condensation reaction is enzyme-catalyzed. For example, the reaction may be catalyzed by a lipase, which can be provided in soluble form or immobilized. In some embodiments, the condensation reaction is a transesterification reaction. Exemplary process for preparing glyceride compositions is described in WO 2019/234057, which is hereby incorporated by reference in its entirety. Suitable lipase enzymes can be derived from a microorganism such as Burkholderia sp., Candida antarctica B, Candida rugosa, Candida cylindracea, Thermomyces lanuginosus, Pseudomonas sp., Candida antarctica A, Porcine pancreas, Humicola sp., Humicola lanuginose, Mucor miehei, Rhizopus javan, Pseudomonas fluor, Pseudomonas cepacia, Candida cylindrcae, Aspergillus niger, Rhizopus oryzae, Mucor jaanicus, Mucor javanicus, Rhizopus sp., Rhizopus japonicus, Rhizomucor miehi, Rhizopus niveus, or penicillium camembertii (also Rhizopus delemar, Pseudonomas aeruginosa). For example, the method may comprise combining an oil (comprising the FAs and BALs in the form of alkyl esters (e.g., ethyl esters), free fatty acids, and/or combinations thereof) with the lipase, and an alcohol (e.g., the molecular backbone such as glycerol) in water. In some embodiments, the lipase is a Candida antarctica lipase B. The reaction can proceed for 2 to about 24 hours, for example, and the reaction can be performed under reduced pressure and sufficient temperature to evaporate ethanol and/or water from the reaction. In some embodiments, the reaction proceeds at a temperature of from about 30° C. to about 90° C. Water can be supplied during at least part of the reaction. The reaction mixture is generally washed one or more times, and then dried. Residual ethyl esters, monoacylglycerols, and/or free fatty acids can be separated from the reaction mixture using known processes.

In still other aspects, the invention provides a method for making a BAL composition for therapeutic delivery. In various embodiments, the method comprises selecting one or a combination of BALs (as described) for therapeutic delivery and then conjugating the BALs to a molecular backbone (as described), and formulating the conjugated BALs as an emulsion (as described) suitable for delivery to a subject in need. In various embodiments, the BALs are selected by performance of the combination in functional assays, and/or animal models.

In another aspect, the invention provides a method for tissue or organ protection in a patient. The method comprises administering an effective amount of a BAL composition described herein, or a BAL composition made by a method described herein, to a patient in need. The invention in various embodiments provides methods for treating a patient in need of protection from cellular death, including acute and chronic injuries to various organs or tissues, such as the brain, spinal cord, heart, lungs, pancreas, kidneys, liver, and intestine, among others. In some embodiments, the patient is in need of treatment for an ischemic organ injury or a traumatic organ injury. In some embodiments, the subject has a chronic condition, involving the degenerative condition of an organ, including but not limited to neurodegenerative conditions. The method generally comprises administering an effective amount of the composition described herein to a patient in need.

In some embodiments, the patient is in need of neuroprotection or cardioprotection. In some embodiments, the patient is at risk of ischemia-reperfusion injury. Ischemia-reperfusion injury is the tissue damage caused when blood supply returns to tissue after a period of ischemia or lack of oxygen. The absence of oxygen and nutrients from blood during the ischemic period creates a condition in which the restoration of circulation results in inflammation and oxidative damage through the induction of oxidative stress.

In some embodiments, the patient has, or had had, or is at risk of myocardial infarction, which is generally triggered when blood flow to the heart muscle is blocked. The composition described herein can be administered after a myocardial infarction, to limit cellular damage to the heart. In other embodiments, the patient has congestive heart failure.

In some embodiments, the patient is experiencing stroke, which can be ischemic stroke or hemorrhagic stroke. For example, cerebral hypoxia-ischemia (or “stroke”) of sufficient duration to deplete high energy reserves in neural cells initiates a cascade of events over the hours to days of reperfusion that culminates in extensive death, both necrotic and apoptotic. These events include the generation of ROS and oxidative damage to cells, release of inflammatory mediators and initiation of prolonged inflammatory reactions, and ongoing apoptosis that can continue for weeks to months.

Thus, in some embodiments the patient is experiencing stroke. Stroke is a major cause of morbidity and mortality through all stages of the life cycle, including for infants born prematurely, for children in intensive care units, and for elderly with cerebral vascular accidents. In some embodiments, the stroke is ischemic stroke. However, the invention also finds use for treating hemorrhagic stroke as well as neonatal stroke. In some embodiments, the subject has or is at risk of hypoxic-ischemic encephalopathy (HIE), which is a type of newborn brain damage caused by oxygen deprivation and limited blood flow. Infants and children who survive HIE demonstrate lifelong neurologic handicaps, including cerebral palsy, mental retardation, epilepsy, and learning disabilities. HIE commonly occurs in critically ill children, most notably in association with cardiopulmonary arrest. The BAL compositions described herein can be administered after stroke onset to provide neuroprotection, that is, inhibit cellular processes leading to cell death.

For example, in various embodiments, the patient is administered the composition within about 1 to about 24 hours of the onset of ischemic injury. For example, in some embodiments, the composition is administered after about 6 hours of the onset of ischemic injury, or after about 8 hours, or after about 10 hours, or after about 12 hours, or after about 15 hours of onset of ischemic or other injury. The composition can prevent substantial cellular death, despite delay in emergency treatment. In some embodiments, the patient is administered the composition within about 2 hours of onset of ischemic injury, or within about 4 hours of the onset of ischemic injury, which provides substantial protection from cell damage and/or death.

The compositions are compatible for treating both ischemic and hemorrhagic stroke, and thus can be administered by emergency personnel, that is prior to brain imaging to detect or visualize the thrombus or potential hemorrhage. In the absence of hemorrhage, the patient may receive thrombolytic therapy to dissolve the clot (e.g., tissue plasminogen activator, t-PA). t-PA catalyzes the conversion of plasminogen to plasmin, the major enzyme responsible for clot breakdown. t-PA is conventionally administered to a stroke victim within about the first 4.5 h after a stroke occurs. In some embodiments, the patient receives such thrombolytic therapy after about 4.5 hours from stroke onset, or after about 6 hours from stroke onset, or after about 8 hours after stroke onset, increasing thrombolytic therapeutic window by delivering t-PA together with DG emulsions. By administering the emulsion compositions as soon as possible in the emergency setting, more time can be obtained to determine whether thrombolytic therapy is appropriate. Thrombolytic therapy cannot be administered for patients experiencing hemorrhagic stroke, since the therapy would exacerbate bleeding.

In still other embodiments, a thrombectomy is performed. Thrombectomy is the interventional procedure of removing a blood clot (thrombus) from a blood vessel. It is commonly performed in the cerebral arteries (interventional neuroradiology). The compositions described herein can expand the window where thrombectomy is successful. For example, the thrombectomy may be performed after about 10 hours from stroke onset, or after about 12 hours from stroke onset. In some embodiments, thrombectomy is performed after about 18 hours or after about 24 hours of stroke onset.

In some embodiments, the patient may receive from 1 to 5 doses of the composition within the first 24 hours, with at least one dose prior to thrombolytic therapy or thrombectomy, and at least one dose after thrombolytic therapy or thrombectomy.

The composition can generally be delivered parenterally, such as intravenously or intra-arterially. In various embodiments, the composition is delivered intravenously or intra-arterially, intrathecally, or by intragastric or intraduodenal tube. In some embodiments, the composition is administered intranasally, allowing for rapid delivery to the brain. In some embodiments, the composition is administered by intra-arterial delivery selectively to the previously hypoperfused brain. In still other embodiments, the composition is administered enterally.

In some embodiments, in the context of ischemic injury (e.g., stroke), the subject may receive a dose of the bioactive lipid composition as soon as possible after the onset of ischemic injury, and generally within about 24 hours, or with about 15 hours, or within about 12 hours, or within about 10 hours, or within about 8 hours, or within about 6 hours of the ischemic injury or reperfusion. The patient may receive subsequent doses over the following days or weeks, to aid recovery. For example, the patient may receive at least 4 administrations of the composition, or may receive at least 8 administrations of the composition. In some embodiments, the patient receives from 1 to 10 or from 1 to 4 administrations over one week to one month following stroke to aid recovery.

In other embodiments, the patient is suffering from or at risk of traumatic brain injury (TBI). TBI usually results from a violent blow or jolt to the head or body. An object that penetrates brain tissue, such as a bullet or shattered piece of skull, also can cause TBI. Mild TBI may affect brain cells temporarily. More-serious TBI can result in bruising, torn tissues, bleeding and other physical damage to the brain. These injuries can result in long-term complications or death. In some embodiments, the patient is administered the composition within 1 to 5 hours of brain injury, or from 1 to 2 hours of brain injury, to reduce long term tissue damage from TBI. In some embodiments, the patient is administered the composition within about 12 hours of brain injury, or within about 24 hours of brain injury. In some embodiments, the patient receives at least 4 administrations of the composition, or may receive at least 8 administrations of the composition. In some embodiments, after the initial administration, the patient is administered the composition at least 4 times or at least 10 times with frequencies ranging from about once every 4 hours to once per week to aid recovery.

In still other embodiments, the patient is suffering from post-traumatic stress disorder (PTSD). PTSD is a serious condition that develops after a person has experienced or witnessed a traumatic or terrifying event in which serious physical harm occurred or was threatened. PTSD is a lasting consequence of traumatic ordeals that cause intense fear, helplessness, or horror, such as a sexual or physical assault, the unexpected death of a loved one, an accident, war, or a natural disaster. In various embodiments, the compositions described herein provide therapeutic value for PTSD. In some embodiments, the patient is administered the composition at least once per week for a period of time to facilitate recovery.

The invention provides use for protecting other organs or tissues, including spinal cord injury (SCI). In such embodiments, the patient may be administered the composition within about 24 hours of injury, or within about 15 hours of injury, or within about 12 hours of injury, or within about 6 hours of injury, or within about 2 hours of injury, or within about 1 hour of injury. In some embodiments, the patient is administered the composition at least once per day or once per week after the initial administration. In some embodiments, the patient receives at least 4 administrations of the composition, or may receive at least 8 administrations of the composition. In some embodiments, after the initial administration, the patient is administered the composition at frequencies ranging from once every 4 hours to once per week (e.g., for at least four weeks) to aid recovery.

Further, in some embodiments, the patient is the recipient of an organ transplant, such as liver, kidney, heart, or lung. In some embodiments, the patient is administered the composition during the perioperative period (e.g., within about 24 hours prior to transplant surgery, and/or within about 24 hours after transplant surgery). In some embodiments, the patient receives at least 4 administrations of the composition, or may receive at least 8 administrations of the composition. In some embodiments, after the initial administration, the patient is administered the composition at frequencies ranging from about once every four hours to about once per week to aid recovery.

In some embodiments, the patient has acute organ failure, such as acute renal, liver, or heart failure. In some embodiments, the patient is administered the composition from 1 to 10 times or from 1 to 4 times with a frequency ranging from about once every 4 hours to once per week to reduce organ damage and/or decline.

In some embodiments, the patient is suffering from a neurodegenerative disease, such amyotrophic lateral sclerosis (ALS), multiple sclerosis (MS), Parkinson's disease, Alzheimer's disease, dementia, and Huntington's disease. For example, the patient is administered the composition at least once per week to slow disease progression, and/or is administered the composition upon disease relapse (e.g., in the case of MS) to reduce the severity and duration of the relapse and/or slow disease progression.

In some embodiments, the patient is administered the composition from 1 to 10 times, with frequencies ranging from about once every four hours to about once per week. In some embodiments, the patient is administered the composition about once daily or about once per week. In some embodiments, the patient is administered the composition at least once per week for at least four weeks.

In these and other embodiments, the patient in need of neuroprotection may in addition, or in some embodiments alternatively, receive oral supplementation with n-3 fatty acids, which can optionally be in the form of DGs or n-3 TGs. Oral supplementation can be administered at least once daily and up to three times daily. Oral supplementation can be provided for one or more weeks or months as needed to support recovery from an acute event, or may be administered indefinitely to aid recovery and prevent relapse or reoccurrence of the condition.

Other aspects and embodiments of the invention will be apparent from the following examples.

### 4. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are described. All publications mentioned herein, are incorporated by reference in their entirety; nothing herein is to be construed as an admission that the invention is not entitled to antedate such disclosure by virtue of prior invention.

### Example 1: Neuroprotective Actions

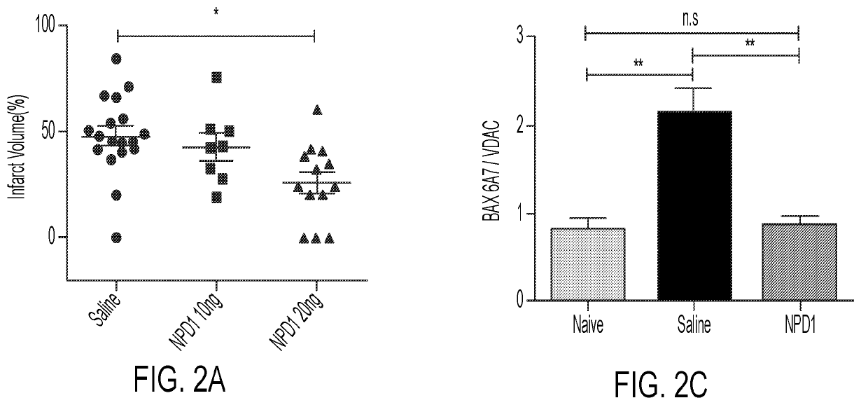

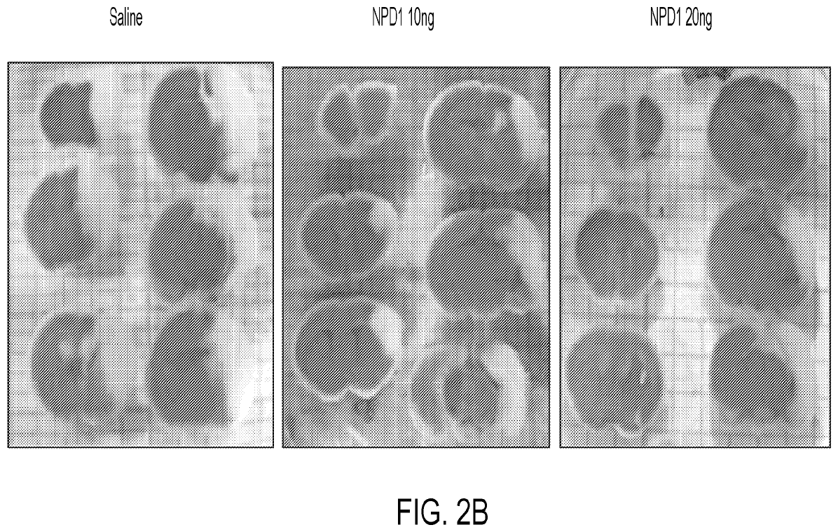

Acute treatment with triglyceride lipid emulsions containing both EPA and DHA or DHA alone (tri-DHA) provides neuroprotection after hypoxic-ischemic brain injury by acting within the initial minutes/hours of reperfusion. There are a number of biological mechanisms affected by omega-3 (n-3) EPA+DHA and tri-DHA emulsions and its bioactive mediators, including (i) decreases in generation of mitochondrial reactive oxygen species (ROS), and (ii) preservation of mitochondrial functions as demonstrated by maintaining mitochondrial Ca2+ uptake and homeostasis. The neuroprotection afforded by tri-DHA acute administration was also associated with increased content of DHA in brain mitochondria and of DHA-derived bioactive mediators, such as neuroprotection D1 (NPD1) and D-series resolvins, in cerebral tissue. These findings led us to also investigate the neuroprotective action afforded by direct acute administration of NPD1 after hypoxic-ischemic injury in a mouse model. We observed that NPD1 treatment induced significant reduction in cerebral infarct volumes compared to the control group, and this was associated with both preserved mitochondrial Ca2+ buffering capacity and reduced mitochondria-related cell death pathways. See FIG. 2.

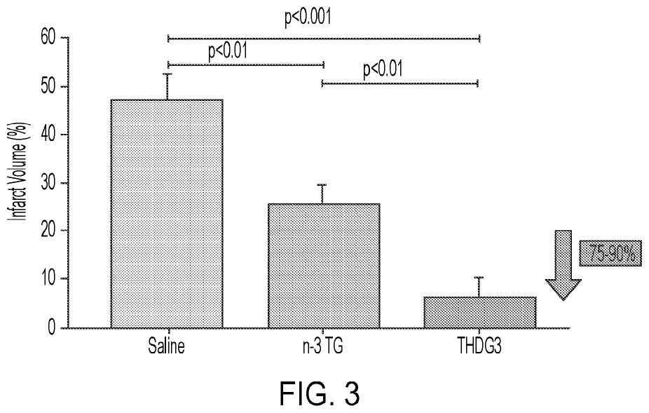

Further, our data show that neonatal mice treated with n-3 fatty acids carried in diglyceride emulsions (containing >90% of total fatty acids as EPA and DHA) exhibited significant reduction in cerebral infarct volumes and n-3 diglyceride emulsions, and were far more effective than n-3 triglyceride emulsions in neonatal HI models. See FIG. 3 and FIG. 4). We characterized the chemical-physical properties of n-3 fatty acids carried in diglyceride emulsions and compared them to triglyceride emulsions. We evaluated the interactions of diglycerides with in vitro models of cellular membranes by NMR spectroscopy. We observed that (i) diglycerides did not adversely affect membrane structures and (ii) incorporated in biological membrane systems more efficiently than triglycerides.

To establish if rapid hydrolysis facilitated clearance of diglyceride emulsions, in vitro lipolysis studies were performed by assessing fatty acid release by lipoprotein lipase (LpL)-mediated hydrolysis. We observed that n-3 diglyceride emulsions had more efficient hydrolysis compared to n-3 triglycerides. This might explain/contribute to the faster uptake and rapid neuroprotection of diglycerides and n-3-related molecules in ischemic brain injury.

We will evaluate whether diglyceride emulsions containing both n-3 fatty acids and other BALs augment the delivery and actions of these molecules in different organs after ischemic injuries. We will investigate cell viability and cell death pathways for screening optimal dose- and cocktail-combinations of different SPMs with n-3. In various aspects the invention offers rapid delivery vehicles for n-3 FAs plus other BALs (such as SPMs), which are expected to show synergistic effects.

### Example 2: Discovery of DG Therapeutics

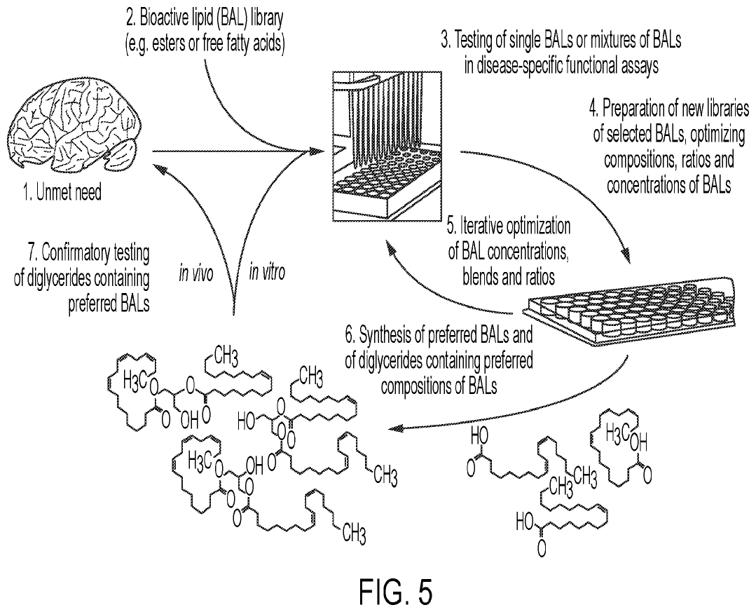

See FIG. 5 for a schematic illustrating a platform for discovery of novel DG therapeutics. Step 1: Identify an unmet medical need for which a DG therapeutic is desired; Step 2: Procure a library of potentially bioactive lipids. The lipids can be procured and tested for example as free fatty acids or esters; Step 3: Testing of lipids in disease-specific functional assays, typically first as single compounds and subsequently as mixtures of bioactive compounds. Steps 4 and 5: The concentrations, compositions and ratios of the lipids are optimized through iterative testing. Before each round of testing, a new library of single compounds or compound mixtures are prepared; Step 6: If needed, the preferred bioactive lipids are first resynthesized and then used to prepare the reaction mixture for lipase-catalyzed synthesis of DG containing the desired BALs in the desired ratios; Step 7: The synthesized DG is emulsified in water and the activity is reconfirmed in in vitro assays and in in vivo models, as desired.

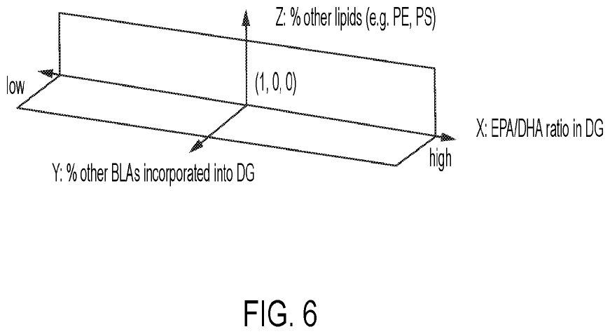

See FIG. 6 for an illustration of the composition of DGs according to embodiments of the invention. The reaction mixture for synthesizing DGs (as shown in FIG. 5) can contain for example both EPA and DHA and in a specific ratio (x-axis). The mixture can be spiked with Specialized Pro-resolving Mediators (SPMs) and other BALs (y-axis), and in some embodiments other types of lipids (z-axis).

### REFERENCES

All references listed below and throughout the specification are hereby incorporated by reference in their entirety.
1. Williams J J, Mayurasakorn K, Vannucci S J, Mastropietro C, Bazan N G, Ten V S, et al. N-3 fatty acid rich triglyceride emulsions are neuroprotective after cerebral hypoxic-ischemic injury in neonatal mice. PLoS One. 2013; 8 (2):e56233.
2. Mayurasakorn K, Niatsetskaya Z V, Sosunov S A, Williams J J, Zirpoli H, Vlasakov I, Deckelbaum R J, Ten V S. DHA but not EPA emulsions preserve neurological and mitochondrial function after brain hypoxia-ischemia in neonatal mice. PLoS One. 2016; 11 (8):e0160870.
3. Zirpoli H, Sosunov S A, Niatsetskaya Z V, Mayurasakorn K, Kollareth D J M, Serhan C N, Ten V S, Deckelbaum R J. NPD1 rapidly targets mitochondria-mediated apoptosis after acute injection protecting brain against ischemic injury. Exp Neurol. 2020 Oct. 7; 113495.

1. A bioactive lipid (BAL) composition comprising one or more BAL which comprises one or more hydrocarbon-derivatized fatty acids (FAs) optionally covalently bound to a molecular backbone.

2. The BAL composition of claim 1, wherein at least one of the BALs is an oxygenated derivative, halogenated derivative, amino derivative, amide derivative, nitro derivative, cyano derivative, furanoid derivative, nitroso derivative, isonitrile derivative, or an O-substituted derivative of the hydrocarbon-derivatized FA.

3. The BAL composition of any one of claims 1-2, wherein at least one of the BALs comprise a specialized pro-resolving mediator (SPMs), eicosanoid, prostanoid, prostaglandin, or endocannabinoid (eCBs) and optionally further comprise omega-3 (n-3) FAs and/or omega-6 (n-6) FAs.

4. The BAL composition of claim 3, wherein the SPM is conjugated or esterified to a molecular backbone and the omega-3 FA is conjugated or esterified to the molecular backbone.

5. The BAL composition of claim 4, wherein at least one SPM is derived from EPA, DHA, DPA, or arachidonic acid.

6. The BAL composition of any one of claims 3-5, wherein at least one SPM is selected from Resolvin D1, Resolvin D2, Resolvin D3, Resolvin D4, Resolvin D5, Resolvin D6, Resolvin E1, Resolvin E2, Resolvin E3, Protectin D1, Resolvin T1, Resolvin T2, Resolvin T3, Resolvin T4, Resolvin 1 n-3 DPA, Resolvin 2 n-3 DPA, Resolvin 5 n-3 DPA, Protectin 1 n-3 DPA, Protectin 2 n-3 DPA, Maresin 1, Maresin 2, Maresin 1 n-3 DPA, Maresin 2 n-3 DPA, and Maresin 3 n-3 DPA, Lipoxin A₄, Lipoxin B₄, or a stereoisomer thereof.

7. The BAL composition of any one of claims 3-5, comprising at least two SPMs independently selected from SPMs derived from EPA, SPMs derived from DHA, and SPMs derived from DPA.

8. The BAL composition of any one of claims 3-5, comprising at least two, at least three, or at least four SPMs independently selected from Resolvin E1, Resolvin E2, Resolvin E3, Resolvin D1, Resolvin D2, Resolvin D3, Resolvin D4, Resolvin D5, Resolvin D6, Protectin D1, Maresin 1, Maresin 2, Resolvin T1, Resolvin T2, Resolvin T3, Resolvin T4, Resolvin 1 n-3 DPA, Resolvin 2 n-3 DPA, Resolvin 5 n-3 DPA, Protectin 1 n-3 DPA, Protectin 2 n-3 DPA, Maresin 1 n-3 DPA, Maresin 2 n-3 DPA, and Maresin 3 n-3 DPA.

9. The BAL composition of claim 3, wherein the prostaglandins are selected from PDG₂, PGE₂, PGI₂, and PGF₂α and the prostanoid is selected from prostacyclin and hromboxane.

10. The BAL composition of claim 3 or claim 9, which further comprises one or more hydroxyeicosatetraenoids (HETEs).

11. The BAL composition of claim 10, wherein the HETEs are selected from leukotrienes and lipoxins (LX).

12. The BAL composition of claim 3 or claim 9, which further comprises epoxyeicosatrienoids.

13. The BAL composition of claim 3, wherein the eCB(s) are selected from arachidonoylethanolamide (AEA), 2-arachidonoylglycerol (2-AG), 2-AG-ether, O-arachidonoylethanolamine, and palmitoylethanolamide (PEA).

14. The BAL composition of claim 3, wherein the BALs comprise at least about 50% n-3 FAs or at least about 75% n-3 FAs.

15. The BAL composition of claim 3 or claim 14, which comprises one or more of docosahexaenoic acid (DHA) and/or eicosapentaenoic acid (EPA) and/or docosapentaenoic acid (DPA) esterified to the molecular backbone.

16. The BAL composition of any one of claims 1-15, wherein the molecular backbone is glycerol.

17. The BAL composition of claim 16, wherein the composition is substantially diglyceride or substantially triglyceride.

18. The BAL composition of any one of claims 1 to 17, wherein the molecular backbone is phosphoglycerol, phosphatidylcholine, phosphatidylserine, phosphatidylinositol, and phosphatidylethanolamine.

19. The BAL composition of any one of claims 1 to 18, wherein the molecular backbone comprises a five-carbon or six-carbon monosaccharide.

20. The BAL composition of any one of claims 1 to 19, wherein the molecular backbone is a polymer, optionally a hydroxyl-containing polymer, and optionally polyethylene glycol, poly(dimethylamine-co-epichlorohydrin, poly(ethyleneglycol) diamine, and/or polyethylenimine or is an amino alcohol.

21. The BAL composition of any one of claims 1 to 20, which is formulated as an emulsion.

22. The BAL composition of claim 21, wherein the composition is an oil-in-water emulsion having a mean particle size of 200 nm or less.

23. The BAL composition of claim 21 or claim 22, wherein at least about 5% by weight of the emulsion is the BAL composition.

24. The BAL composition of any one of claims 21 to 23, wherein the emulsion comprises one or more emulsifiers.

25. The BAL composition of claim 24, wherein the emulsifier is selected from one or more of a phospholipid emulsifier, a phosphatidylcholine emulsifier and one or more medium chain or long chain FAs emulsifiers.

26. The method for making the BAL composition of any one of claims 1 to 25, comprising, performing a condensation reaction with the molecular backbone and one or more BAL.

27. The method of claim 26, wherein the condensation reaction is enzyme-catalyzed, optionally with a lipase.

28. The method of claim 26, wherein the condensation reaction is an esterification or transesterification reaction.

29. The method of claim 26, comprising:
selecting a combination of BALs for therapeutic delivery;
conjugating the BALs to a molecular backbone to form a BAL composition; and
formulating the BAL composition as an emulsion suitable for delivery to a subject in need.

30. The method of claim 26, wherein the BALs are selected by their performance in one or more cellular assays selected from one or more of evaluating caspase-3/7 activation, MTT cell viability assay, lactate dehydrogenase (LDH) assay, TUNEL assay, Annexin V/propidium iodide (PI) assay, detection of proteins associated with apoptosis or cell death, cytokine detection, assays for oxidative stress or ROS production, assays for inflammasome activation, expression or production of mitochondrial biomarkers, measurement of intracellular Ca2+, and/or one or more animal models oxygen and glucose deprivation (OGD) in vitro, selected from middle cerebral artery occlusion (MCAo), hypoxic-ischemic injury, LPS-induced cell inflammation in vitro, traumatic brain injury, left anterior descending artery (LAD) occlusion, global heart ischemia ex vivo system, and spinal cord injury in vivo.

31. A method for tissue or organ protection in a patient, comprising administering an effective amount of the composition of any one of claims 1-25 to a patient in need.

32. The method of claim 31, wherein the patient is at risk of ischemia reperfusion injury, the patient is experiencing stroke, the patient is recovering from stroke, the patient is at risk of stroke, the patient has hypoxic-ischemic encephalopathy, the patient is at risk for hypoxic-ischemic encephalopathy, the patient is experiencing myocardial infarction, the patient is recovering from myocardial infarction, the patient is at risk of myocardial infarction, the patient is suffering from traumatic brain injury, the patient is at risk of traumatic brain injury, the patient is suffering from post-traumatic stress disorder, the patient is suffering from a spinal cord injury, the patient is suffering from a neurodegenerative disease, the patient is suffering from acute organ injury, or the patient is an organ transplant recipient.

33. The method of claim 32, wherein the stroke is ischemic stroke, hemorrhagic stroke, or neonatal stroke.

34. The method of claim 32, wherein the neurodegenerative disease is amyotrophic lateral sclerosis, multiple sclerosis, Parkinson's disease, Alzheimer's disease, Dementia, or Huntington's disease.

35. The method of any one of claims 31-34, wherein the composition is administered intravenously, intra-arterially, intrathecally, or by intragastric or intraduodenal tube.

36. The method of any one of claims 31-35, wherein the patient is administered the composition from 1 to 10 times, with frequencies ranging from about once every four hours to about once per week.
